(19) United States
(12) Patent Application Publication
(10) Pub. No.: US 2011/0071354 A1
(43) Pub. Date: Mar. 24, 2011

(54) OVERTUBE AND MEDICAL PROCEDURE VIA NATURAL ORIFICE USING THE SAME

(75) Inventors: Manabu Miyamoto, Tokyo (JP); Takahiro Kogasaka, Tokyo (JP); Takumi Dejima, Tokyo (JP); Kiyotaka Matsuno, Sagamihara-shi (JP); Ken Yamatani, Tokyo (JP); Saori Takeuchi, Tokyo (JP)

(73) Assignee: OLYMPUS MEDICAL SYSTEMS CORP., TOKYO (JP)

(21) Appl. No.: 12/958,867

(22) Filed: Dec. 2, 2010

Related U.S. Application Data
(63) Continuation of application No. 11/435,182, filed on May 16, 2006.
Application No. 11/435,182 is a continuation-in-part of application No. 11/331,938, filed on Jan. 13, 2006, now abandoned.

(51) Int. Cl. A61B 1/00

(52) U.S. Cl. 600/114

(57) ABSTRACT

An overtube according to this invention includes: an insertion part, that is inserted into a subject and has a lumen, through which a device insertion part of a device for performing a medical procedure inside a body of the subject is removably inserted, the insertion part being inserted into the subject; and a tissue incising part that is disposed at a distal end side of the insertion part so as to cross the lumen and incises a tissue of the subject.

### CROSS REFERENCE TO RELATED APPLICATIONS

The present application is a continuation of U.S. patent application Ser. No. 11/435,182, filed on May 16, 2006 which is a continuation-in-part application of U.S. patent application Ser. No. 11/331,938, filed on Jan. 13, 2006, the contents of which were entirely incorporated herein by reference.

### BACKGROUND OF THE INVENTION

1. Field of the Invention

This invention relates to an overtube and a medical procedure using the overtube that is performed via a natural orifice.

2. Description of Related Art

Laparoscopic operations are known in which, in performing a medical procedure of observing, treating, etc. an organ of the human body, instead of incising the abdominal wall widely, a plurality of orifices are opened in the abdominal wall and procedures are performed upon inserting a laparoscope, forceps, and other treatment instruments into the orifices. Such procedure provides the benefit of lessening the burden placed on the patient because only small orifices need to be opened in the abdominal wall.

In recent years, methods of performing procedures upon inserting a flexible endoscope via the mouth, nose, anus, or other natural orifice of the patient have been proposed as methods of further reducing the burden on the patient. An example of such procedures is disclosed in U.S. Pat. No. 5,458,131.

With this method, a flexible endoscope is inserted from the mouth of a patient, an opening is formed in the stomach wall, and a distal end part of the endoscope is fed into the abdominal cavity from the opening. Then while using the endoscope as a device for observing the interior of the abdominal cavity, desired procedures are performed inside the abdominal cavity using a treatment instrument inserted through the endoscope or a treatment instrument inserted from another opening.

### SUMMARY OF THE INVENTION

An object of this invention is to provide a device and a method that enable incision of tissue to be performed more readily in performing a medical procedure using an overtube.

An overtube according to a first aspect of this invention includes: an insertion part, that is inserted into a subject and has a lumen, through which a device insertion part of a device for performing a medical procedure inside a body of the subject is removably inserted, the insertion part being inserted into the subject; and a tissue incising part that is disposed at a distal end side of the insertion part so as to cross the lumen and incises a tissue of the subject.

An overtube according to a second aspect of this invention includes: an insertion part, that is opened at a distal end, inserted into a subject, and has a lumen, through which a device insertion part of a device for performing a medical procedure inside a body of the subject is removably inserted, the insertion part being inserted into the subject; and a tissue incising part that crosses a distal end side of the lumen is disposed at the insertion part so as to allow withdrawing of the crossing state, and incises a tissue of the subject.

A medical procedure through a natural orifice according to a third aspect of this invention includes: inserting a device that extends in an axial direction into a lumen disposed in an insertion part of an overtube and inserting the insertion part into a hollow organ through a natural orifice of a subject; guiding the insertion part to an incision target site while using an observation device to observe the incision target site; using a tissue incising part, disposed at a distal end side of the insertion part, to incise the incision target site and form an opening; and introducing at least one of an operative device and the overtube into an abdominal cavity via the opening.

### BRIEF DESCRIPTION OF THE DRAWINGS

FIG. 1 is a schematic view of an entirety of an overtube according to a first embodiment.

FIG. 2 is a view of principal portions of the overtube according to the first embodiment.

FIG. 3 is a perspective view of a distal end part of the overtube according to the first embodiment.

FIG. 4A is a sectional view taken along line III-III of FIG. 3.

FIG. 4B is a sectional view taken along line IV-IV of FIG. 4A.

FIG. 5 is a partially enlarged section of an electrode manipulating part of the overtube according to the first embodiment.

FIG. 6A is an entire view of double T-bars used in the embodiment.

FIG. 6B is a sectional view of a state in which the double T-bars are fitted into a puncture needle of the overtube according to the first embodiment.

FIG. 7 is a partial sectional view of a needle manipulating part of the overtube according to the first embodiment.

FIG. 8 is a sectional view of a portion near an endoscope lock button of the overtube according to the first embodiment.

FIG. 9 is an entire schematic view of an endoscope as an example of a device used for the overtube according to the first embodiment.

FIG. 10 is a flowchart of a medical procedure according to the first embodiment.

FIG. 11 is a view for describing a state of inserting the endoscope into the overtube in the medical procedure according to the first embodiment.

FIG. 12 is a view for describing a state of introducing the overtube to an incision target site in the medical procedure according to the first embodiment.

FIG. 13 is a view for describing a state of suctioning a portion of a stomach wall into the overtube in the medical procedure according to the first embodiment.

FIG. 14 is a view for describing a state of puncturing the suctioned stomach wall by means of the puncture needle of the overtube in the medical procedure according to the first embodiment.

FIG. 15 is a view for describing a state of insufflation of an abdominal cavity by feeding of air from a hypodermic needle in the medical procedure according to the first embodiment.

FIG. 16 is a view for describing a state of releasing anchors of the double T-bars from the puncture needle in the medical procedure according to the first embodiment.

FIG. 17 is a view for describing a state of incising the suctioned stomach wall by means of the incising electrode of the overtube in the medical procedure according to the first embodiment.

FIG. 18 is a view of FIG. 17 viewed from a direction rotated by 90 degrees.

FIG. 19 is a view for describing a state of inserting the endoscope into the abdominal cavity in the medical procedure according to the first embodiment.

FIG. 20 is a view for describing a state of pulling and constricting a string of the double T-bars that have been placed in the medical procedure according to the first embodiment.

FIG. 21 is a view of principal portions of an overtube according to a second embodiment.

FIG. 22 is a sectional view taken along line A-A of FIG. 21.

FIG. 23 is a flow chart of a medical procedure according to the second embodiment.

FIG. 24A is a view for describing a state of making an endoscope inserting part protrude from the overtube in the medical procedure according to the second embodiment.

FIG. 24B is a view for describing a state of using the endoscope inserting part as a guide and using a second magnet of the overtube to move the overtube inside an abdominal cavity from the state shown in FIG. 24A.

FIG. 25A is a view for describing a state of making the endoscope inserting part protrude along with the overtube from an orifice in the medical procedure according to the second embodiment.

FIG. 25B is a view for describing a state of using the second magnet of the overtube to move the endoscope inserting part and the overtube inside the abdominal cavity from the state shown in FIG. 25A.

FIG. 26 is a view for describing a state of using the first magnet of the overtube to support the overtube inside the abdominal cavity in the medical procedure according to the second embodiment.

FIG. 27 is a view for describing a state of using the first magnet and third magnet of the overtube to change the direction of the overtube inside the abdominal cavity in the medical procedure according to the second embodiment.

FIG. 28 is a perspective view of a modification example of principal portions of an overtube.

FIG. 29 is a perspective view of another modification example of principal portions of an overtube.

FIG. 30 is an entire schematic view showing a modification example of the overtube according to the first embodiment.

FIG. 31 is a perspective view showing the distal end part in a modification example of the overtube according to the first embodiment.

FIG. 32 is a cross-sectional view showing the state in which the double T-bar has been attached to the puncture needle in a modification example of the overtube according to the first embodiment.

FIG. 33 is a cross-sectional view showing the state in which the double T-bar has been attached to the puncture needle in a modification example of the overtube according to the first embodiment.

FIG. 34 is a cross-sectional view showing the state after puncturing with the puncture needle in a modification example of the overtube according to the first embodiment.

FIG. 35 is an entire schematic view showing another modification example of the overtube according to the first embodiment.

FIG. 36 is a perspective view showing the distal end part in another modification example of the overtube according to the first embodiment.

### DETAILED DESCRIPTION OF THE INVENTION

Embodiments according to the present invention will now be described in detail below. In the following description, components that are the same shall be provided with the same numeric symbol and redundant description shall be omitted.

### First Embodiment

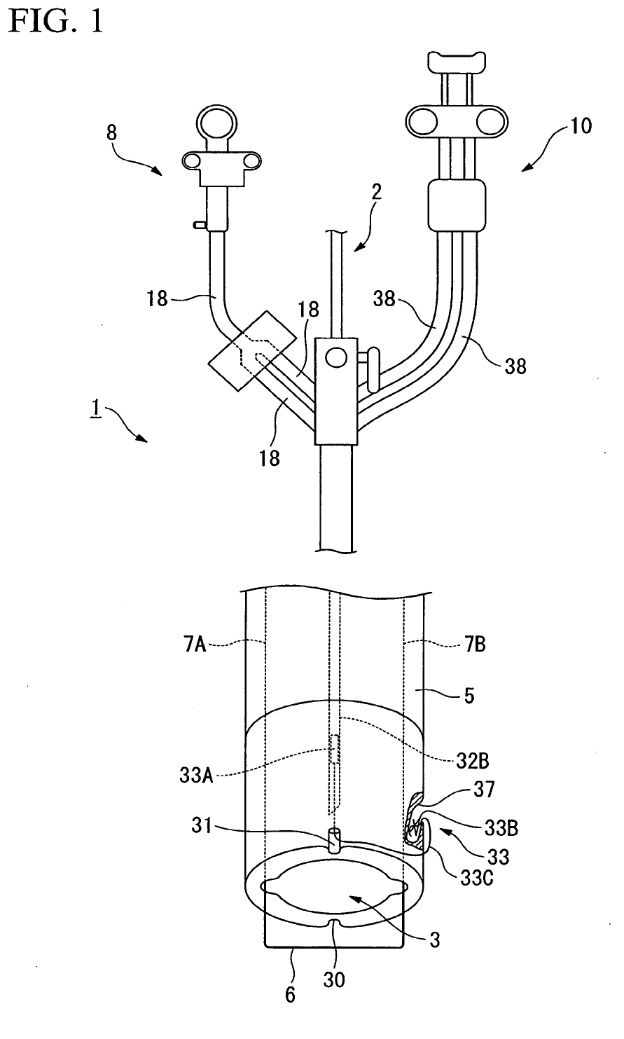

An overtube 1 according to this embodiment is used as a guide tube for inserting, into a body, an endoscope or other device, for carrying out a medical procedure inside a body and being equipped with an insertion device part that is inserted inside a subject (to simplify the description, the device may be referred to simply as “device” or “endoscope” below). As shown in FIG. 1, the overtube 1 includes: an insertion part 5 that is opened at a distal end, inserted into a stomach or other hollow organ or abdominal cavity, etc., of a patient (subject) and has a lumen 3, through which an endoscope 2, as one example of a device extending along an axial direction, is removably inserted; an incising electrode (tissue incising part) 6 that crosses a distal end side of the lumen 3, is disposed in the insertion part 5 so as to allow withdrawing of the crossing state, and incises an internal tissue of the patient; electrode manipulating wires (manipulating members) 7A and 7B that are each connected to the incising electrode 6 and are disposed in a manner enabling advancing and retracting with respect to the insertion part 5; an electrode manipulating part (manipulating part) 8 that is for manipulating the electrode manipulating wires 7A and 7B to advance and retract with respect to the lumen 3; and a needle manipulating part 10 that is for manipulating a puncture needle 32A, 32B, and pusher 35 to be described later. Though in the present embodiment, the incising electrode 6 and the electrode manipulating wires 7A and 7B are arranged from a single wire, the incising electrode 6 and electrode manipulating wires 7A and 7B may be arranged as separate members that are connected to each other. Also, the incising electrode 6 may be disposed so as not to be able to advance and retract with respect to the insertion part 5.

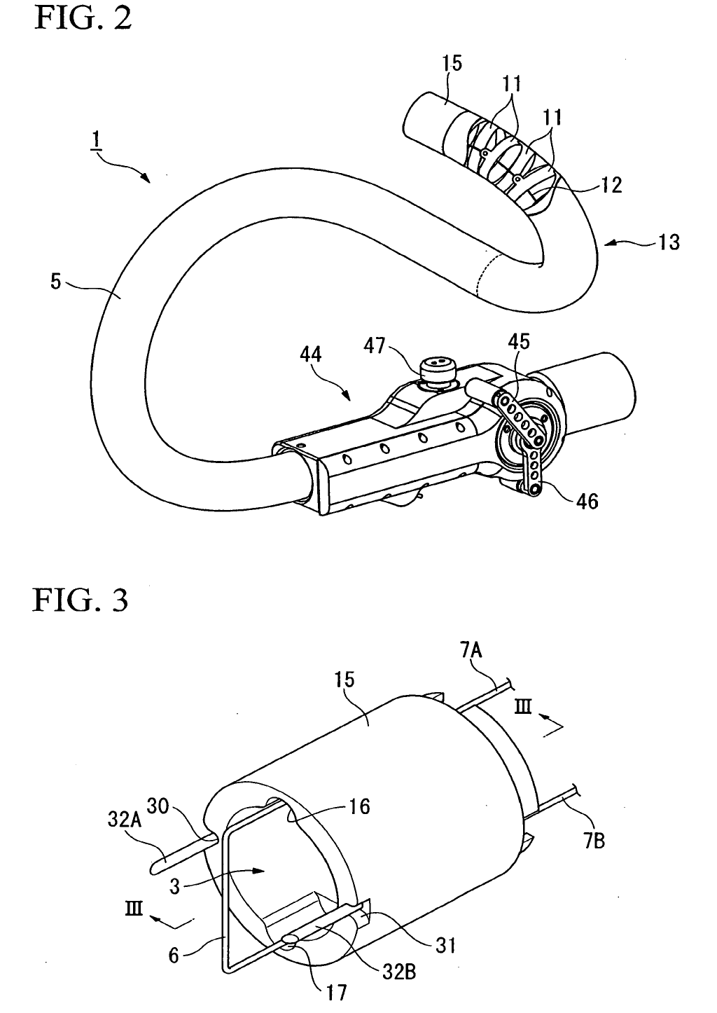

As shown in FIG. 2, the insertion part 5 is elongated and flexible, and as with a normal flexible endoscope, a bending part 13, in which a plurality of joint rings 11 are connected along bending wires 12, is disposed at a distal end side of the insertion part 5. Here, instead of providing the bending part 13 that is actively bended by manipulation by an operator, the insertion part may be arranged in a tube-like shape with flexibility and to be bended passively in accordance with a bending state of the endoscope, etc. A distal end part 15 having a short pipe shape is disposed further at the distal end of the bending part 13 as shown in FIG. 3.

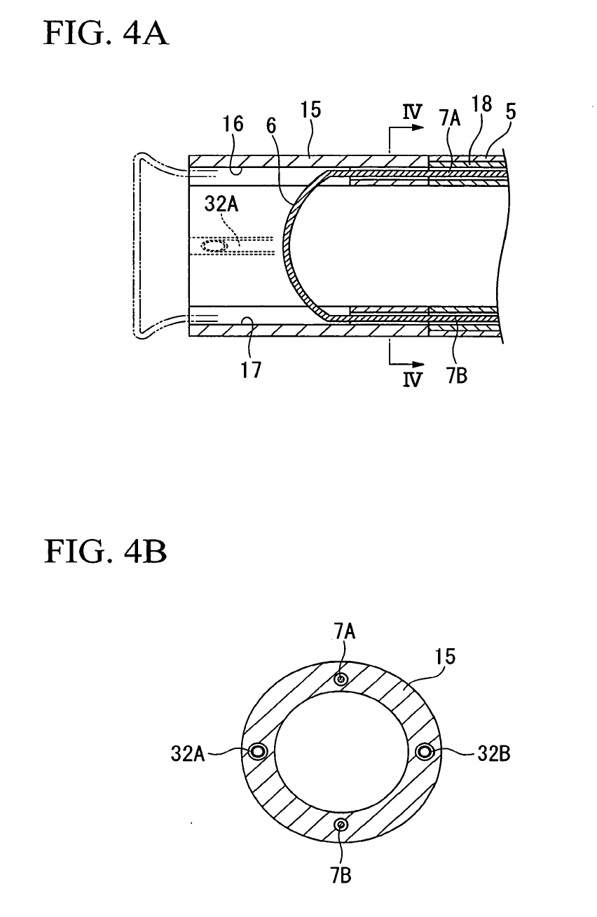

The incising electrode 6 is, for example, a stainless steel wire to which high-frequency electricity can be energized and is disposed so as to cross a center of the lumen 3 in a direction orthogonal to an axial direction of the insertion part 5. That is, as shown in FIGS. 3 and 4, one end side of the incising electrode 6 is inserted through a first inner groove 16, formed on an outer edge of the lumen 3 that is an inner surface of the distal end part 15 (in other words, an inner periphery of the distal end part 15 that defines the lumen 3), and is connected to the electrode manipulating wire 7A. The other end side of the incising electrode 6 is inserted through a second inner groove 17, formed on the outer edge of lumen 3 (in other words, the inner periphery of the distal end part 15 that defines the lumen 3) at a position substantially symmetrically across the center of the lumen 3 from the first inner groove 16, and is connected to the electrode manipulating wire 7B. The incising electrode 6 is formed so that its length is longer than the inner diameter of the lumen 3, and as shown in FIG. 4, the incising electrode 6 is accommodated in the lumen 3 with a bended state, and is movable along the first inner groove 16 and second inner groove 17. Though in the embodiment shown in FIG. 4, the length of the incising electrode 6 is set longer than the inner diameter of the lumen 3, this invention is not restricted thereto, and the length of the incising electrode 6 may be set (for example, to a length substantially equal to the inner diameter of the lumen 3) so that the incising electrode 6 is accommodated in the lumen 3 with a non-bended state.

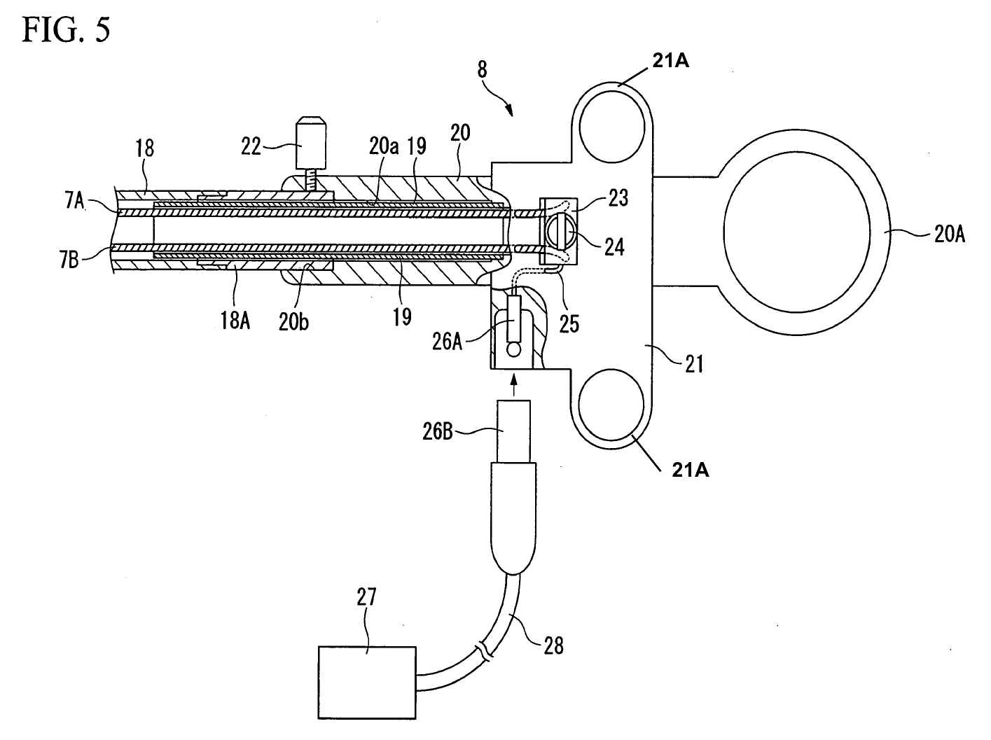

The electrode manipulating wires 7A and 7B are inserted through an electrode tube 18. The electrode tube 18 is provided with a single tube at a proximal end side that protrudes outside the insertion part 5. As shown in FIG. 1, the electrode tube 18 branches into two at a middle portion, and are connected at a distal end to the distal end part 15, so that the electrode manipulating wires 7A and 7B are accommodated separately in the insertion part 5. As shown in FIG. 5, the proximal ends of the electrode manipulating wires 7A and 7B are inserted through a single, rigid manipulating pipe 19 disposed to protrude from a distal end of a manipulating handle 21 to be described later.

As shown in FIGS. 1 and 5, the electrode manipulating part 8 includes a manipulating body 20, which is connected to the proximal end of the electrode tube 18, and a manipulating handle 21, which is disposed to be able to advance and retract freely with respect to the manipulating body 20. The manipulating body 20 is provided with an insertion hole 20a, through which the electrode manipulating wires 7A and 7B and the manipulating pipe 19 are inserted. At a distal end of the manipulating body 20 is formed an engagement hole 20b, which engages with a rigid part 18A disposed at the proximal end of the electrode tube 18, and the electrode tube 18 is fixed to the engagement hole 20b by a screw 22. A finger ring 20A is disposed at a proximal end of the manipulating body 20.

A connection plate 23 is disposed at the manipulating handle 21. The connection plate 23 is electrically connected to end parts of the electrode manipulating wires 7A and 7B inserted through the manipulating pipe 19. A fixing screw 24 is disposed at the connection plate 23, and by screwing the fixing screw 24 into the connection plate 23, the electrode manipulating wires 7A and 7B are fixed and electrically connected. The connection plate 23 is electrically connected via an electric wiring 25 to a connection terminal 26A disposed in the manipulating handle 21. A connection terminal 26B, disposed at a distal end of a power supply cord 28 that extends from a high-frequency power supply 27, is detachably attached to the connection terminal 26A. The manipulating handle 21 is also provided with finger rings 21A.

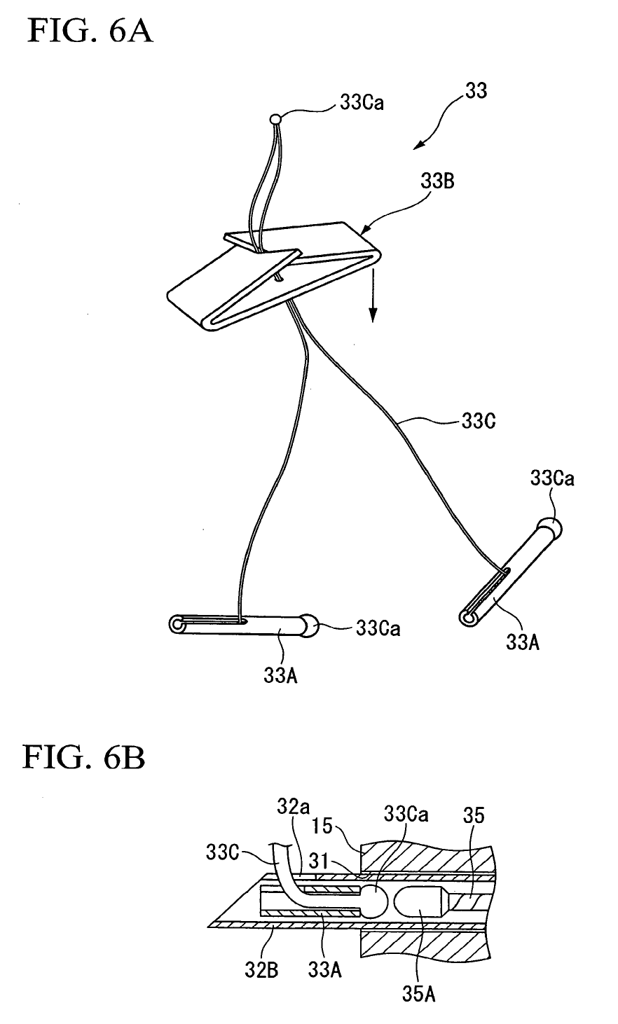

On an outer surface of the distal end part 15 of the overtube 1, a first outer groove 30 and a second outer groove 31 are formed from a middle portion to the distal end of the distal end part 15 at positions orthogonal to a direction joining the first inner groove 16 and the second inner groove 17. The two puncture needles (hollow needles) 32A and 32B, which advance and retract along the lumen 3, are movably disposed in advancing and retracting directions in the first outer groove 30 and the second outer groove 31, respectively. Two anchors 33A of double T-bars 33, shown in FIG. 6A, are respectively held inside the respective puncture needles 32A and 32B as shown in FIG. 6B.

The double T-bars 33 have two sutures 33C, one end side of each of which is passed through a substantially triangular stopper 33B. At one end, the sutures 33C are bound together to form a large diameter part 33Ca. Each of the other ends of the sutures 33C is fixed to the anchors 33A. Each anchor 33A has a cylindrical shape with a slit formed at an end, and the suture 33C is inserted in the longitudinal direction of the interior of anchor 33A through the slit. The large diameter part 33Ca that has greater diameter than that of the anchor 33A is formed at the other end of the suture 33C. The stopper 33B has a hole, through which the sutures 33C are passed, at a center in the longitudinal direction of an elongated, thin plate member. The respective ends in the longitudinal direction of the stopper 33B are folded obliquely and sandwich the sutures 33C. The respective ends in the longitudinal direction of the stopper 33B are cut to notches of triangular shape. With the stopper 33B, the respective ends are folded back obliquely so that the notches intersect and thereby sandwich the sutures 33C. The sutures 33C thus do not fall off from between the ends. When the large diameter part 33Ca of the sutures 33C is pulled in a direction away from the stopper 33B, the respective end parts of the stopper 33B spread apart slightly. The stopper 33B thus allows movement of the sutures 33C in this direction. Meanwhile, when a large diameter part 33Ca at the anchor 33A side of a suture 33C is pulled, a tendency for the suture 33C to move in the direction indicated by the arrow in FIG. 6A arises. However, since the respective ends of the stopper 33B close and grasp the sutures 33C in this process, the suture 33C does not move.

As shown in FIG. 6B, the pusher 35 is movably disposed in advancing and retracting directions in the interior of the respective puncture needles 32A and 32B. As shown in FIG. 1, the stopper 33B of the double T-bars 33 is accommodated inside a hole (receiving part) (referred to hereinafter simply as “hole”) 37 formed from a proximal end side to the distal end side of a side face of the insertion part 5.

The puncture needles 32A and 32B and pushers 35 are respectively accommodated in two outer sheaths 38. Each of the two outer sheaths 38 is inserted through the insertion part 5 and has a distal end connected to the distal end part 15. A slit 32a, through which a suture 33C of the double T-bars 33 is inserted, is formed at a distal end of each of the puncture needles 32A and 32B. A rigid, pushing member 35A is disposed at a distal end of the pusher 35.

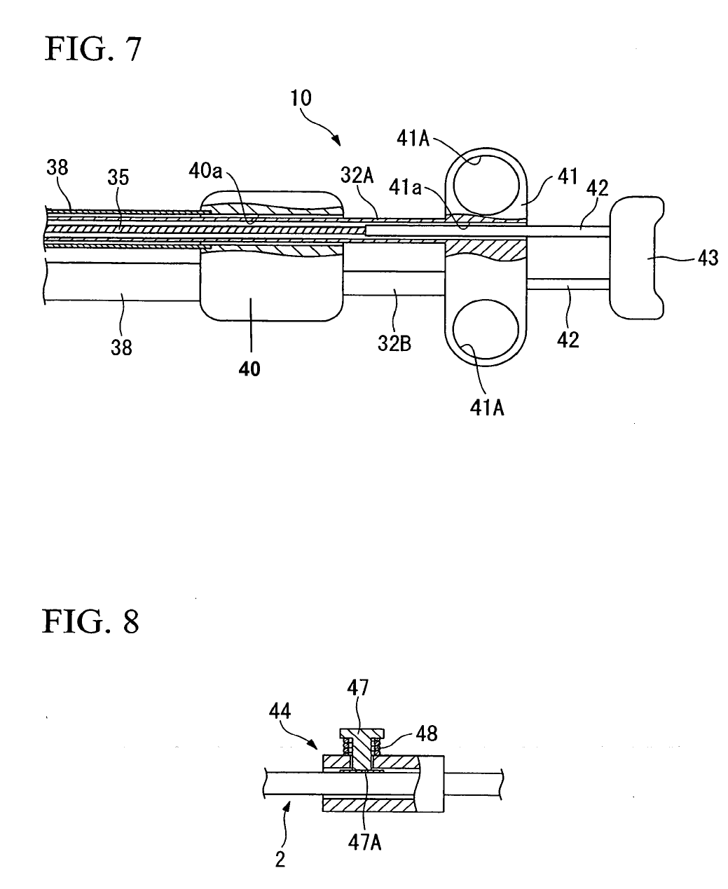

As shown in FIGS. 1 and 7, the needle manipulating part 10 includes: a sheath holding part 40, connected to the proximal ends of the two outer sheaths 38; a needle manipulating handle 41, connected to proximal ends of the two puncture needles 32A and 32B that have been passed in a manner enabling advancing and retracting through through-holes 40a formed in the sheath holding part 40; and a pusher connection part 43 that connects end portions of rod-like, rigid parts 42, which are passed in a manner enabling advancing and retracting through through-holes 41a formed in the needle manipulating handle 41 and are connected to proximal ends of the two pushers 35, to each other. The needle manipulating handle 41 is provided with finger rings 41A. Each of the needle manipulating handle 41 and the pusher connection part 43 may be divided into two parts so as to enable the two puncture needles 32A and 32B and the two pushers 35 to be manipulated independently of each other.

As shown in FIG. 2, a proximal handle 44 having a larger diameter than the insertion part 5, is disposed at the proximal end of the insertion part 5 of the overtube 1. The proximal handle 44 includes a bending lever 45, a bending lock lever 46, and an endoscope lock button 47. The bending lever 45 is connected to the proximal ends of the bending wires 12 for performing bending manipulation of the bending part 13. The bending lock lever 46 is used for fix the position of the bending lever 45 at an arbitrary position. The endoscope lock button 47 is used for fix the endoscope 2 with respect to the lumen 3 upon insertion of the endoscope 2 through the lumen 3.

The distal ends of the bending wires 12 are fixed to the distal end part 15, and in the present embodiment, the two bending wires 12 are inserted through the interior of the insertion part 5 and the distal ends thereof are fixed to portions of the distal end part 15 that substantially oppose each other across the center of the lumen 3. Though in this embodiment, two bending wires 12 are provided to enable bending of the bending part 13 in two directions, this invention is not limited thereto, and four bending wires 12 and two bending levers 45 may be provided as in a bending part of a known endoscope to enable bending of the bending part in four directions.

As shown in FIG. 8, the endoscope lock button 47 has a pressing part 47A of wide width disposed at a distal end and which is normally urged in an outward radial direction of the proximal handle 44 by a spring 48. When the endoscope 2 must be fixed to the insertion part 5 upon being inserted through the interior, the endoscope lock button 47 is pressed inward in the radial direction so that the pressing part 47A presses and fixes the endoscope 2 in a relative manner by a frictional force. The endoscope lock button 47 may be arranged so as to oppositely release the frictional force when pressed.

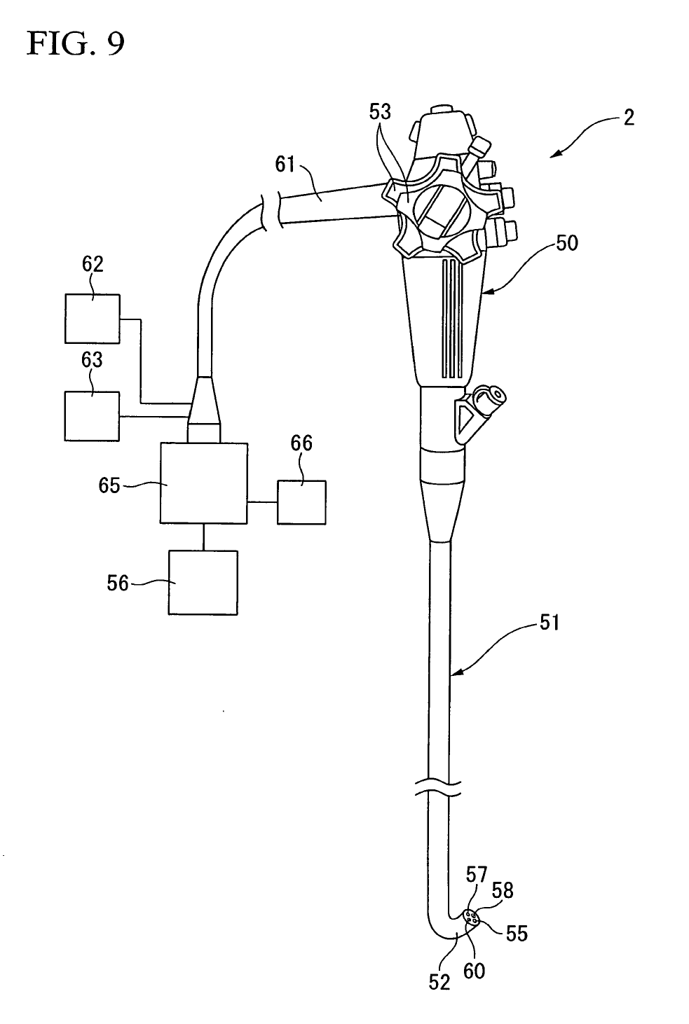

The endoscope 2, which is inserted into the overtube 1, is, for example, a flexible endoscope and, as shown in FIG. 9, an endoscope inserting part 51, which is elongated and inserted into a patient's body, extends outward from an endoscope manipulating part 50 manipulated by an operator. An endoscope distal end part 52 of the endoscope inserting part 51 can be bended by manipulating an angle knob 53 disposed at the endoscope manipulating part 50. At the endoscope distal end part 52 are disposed an objective lens 55, a distal end face of an optical fiber 57 that guides light from a light source device 56 disposed outside the body, and distal end openings of channels 58 and 60. The channel 58 is a duct that is connected via a universal cable 61 to an air/water feeding device 62 or a suction device 63 disposed outside the body and is used to supply or drain fluid to or from inside the body. The channel 60 is a duct for inserting and removing a treatment instrument and is disposed at a position of six o'clock to eight o'clock of the endoscope inserting part 51. The number of treatment instrument channels is not restricted to one and, for example, two treatment instrument channels may be provided. An observation image inputted into the objective lens 55 is displayed on a monitor 66 via a control unit 65.

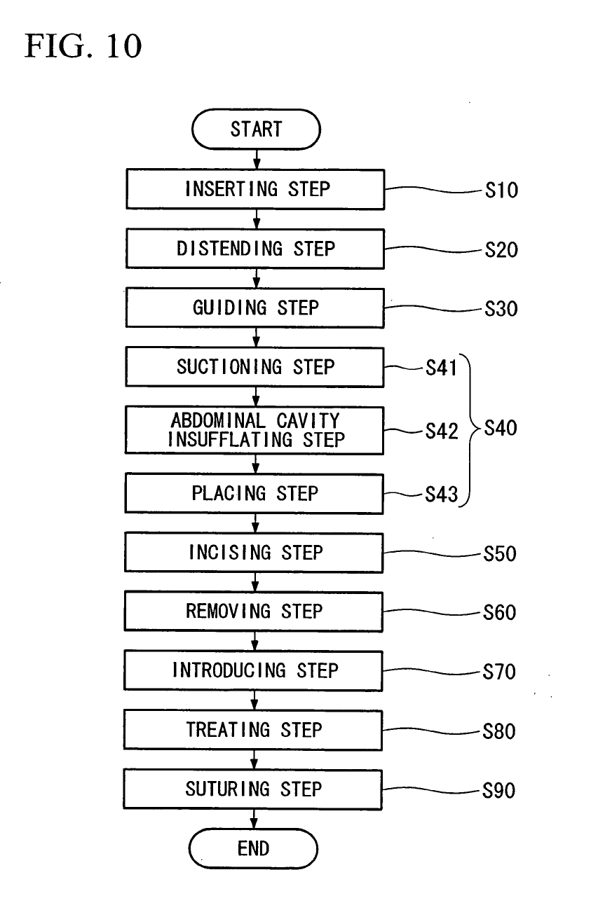

Actions of the present embodiment shall now be described in line with a medical procedure performed via a natural orifice using the overtube 1 as shown by the flow chart of FIG. 10. In the following description, it shall be deemed that an incision target site T is located on an anterior wall of a stomach ST, and a surgical procedure of inserting the endoscope 2 into the stomach (hollow organ) ST from a mouth M of a patient PT and performing treatment upon forming an opening SO in the stomach wall and inserting the insertion part 5 of the endoscope 2 into an abdominal cavity AC shall be described. Also, though in the embodiment described below, the endoscope 2 is introduced as a device into the body from the mouth M of the patient PT and made to approach the abdominal cavity AC upon forming the opening SO in the anterior wall of the stomach ST, the natural orifice from which the endoscope 2 is introduced is not restricted to the mouth M and may be another natural orifice, such as the anus, nose, etc. Furthermore, though the forming of the opening SO in the anterior wall of the stomach ST is desirable, this invention is not restricted thereto, and an opening may be formed on a wall of the esophagus, small intestine, large intestine or other hollow organ (hollow organ) besides the stomach ST into which a device is introduced via a natural orifice.

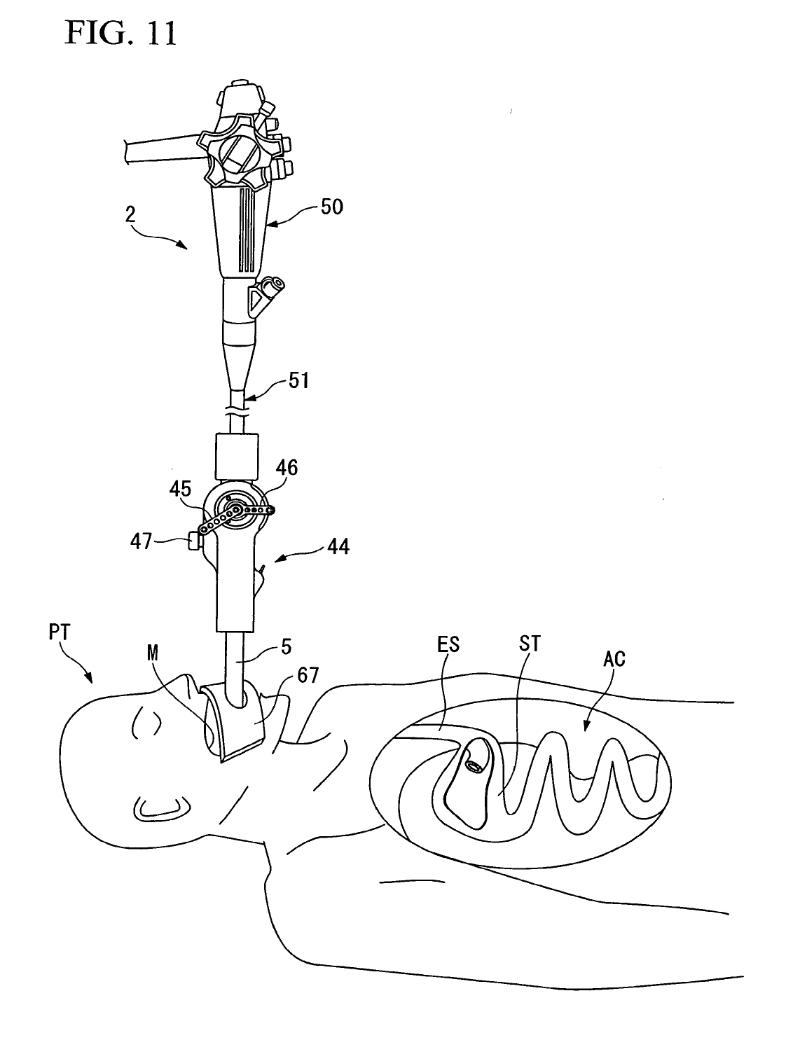

First, with the patient PT being made to lie in a supine position, an inserting step (S10) of inserting the endoscope 2 through the lumen 3 in the insertion part 5 of the overtube 1 and inserting the insertion part 5 of the overtube 1 and the endoscope 2 into the stomach (hollow organ) ST from the mouth M of the patient PT while observing the interior of the body cavity by means of an endoscopic image is performed. As shown in FIG. 11, a mouthpiece 67 is fitted onto the mouth of the patient PT and the overtube 1 and the endoscope 2 are inserted, with the endoscope 2 being inserted through the interior of the lumen 3, into the esophagus ES from the mouthpiece 67. It shall be deemed that the incising electrode 6 and the puncture needles 32A and 32B are all accommodated and positioned at initial positions inside the distal end part 15.

Next, in a distending step (S20), air is supplied from the air/water feeding device 62 via the channel 58 of the insertion part 5 to inflate the stomach ST.

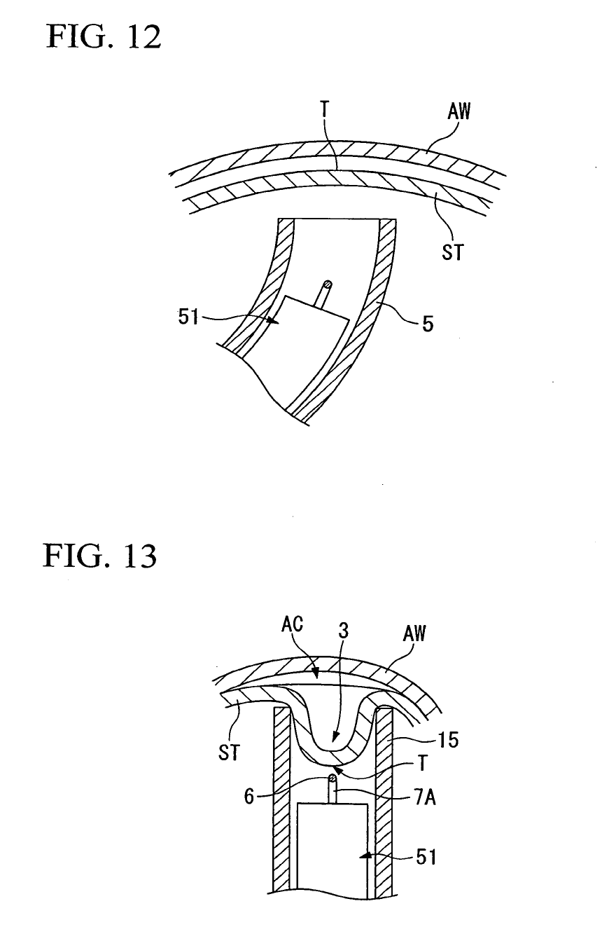

A guiding step (S30) of guiding the insertion part 5 of the overtube 1 to the incision target site T while checking the incision target site T using the endoscope 2, which is also an observation device, is then performed. First, after inserting the endoscope inserting part 51 of the endoscope 2 into the stomach ST, the angle knob 53 is manipulated to bring the distal end of the endoscope inserting part 51 close to the incision target site T while observing the interior of the stomach ST via the objective lens 55, disposed at the endoscope inserting part 51. Then with the incision target site T being specified, the endoscope inserting part 51 is used as a guide to push the insertion part 5 of the overtube 1 and bring the distal end part 15 of the overtube 1 close to the incision target site T as shown in FIG. 12.

A needle moving step (S40), of advancing and retracting the puncture needles 32A and 32B, disposed at the distal end side of the insertion part 5, along the lumen 3, is then performed. First, in a suctioning step (S41), a stomach wall that includes the incision target site T is sucked in by the suction device 63 via the channel 58, with the distal end part 15 being put in contact with the stomach wall. In this process, a portion of the stomach wall is sucked into the distal end part 15 as shown in FIG. 13. A space is thereby secured between an outer side of the stomach wall and the abdominal cavity AC. The means for suctioning the stomach wall is not restricted to the method of using the channel 58 of the endoscope 2. For example, a space, formed between an inner surface of the lumen 3 of the overtube 1 and an outer periphery of the insertion part 5 of the endoscope 2 or other device inserted into the lumen 3, may be used as a suction channel and suction may be performed upon connecting the channel to the suction device 63. In this case, a valve (not shown) that controls the flow of fluid between the interior and exterior of the body may be provided in the formed space to further improve the suction effect.

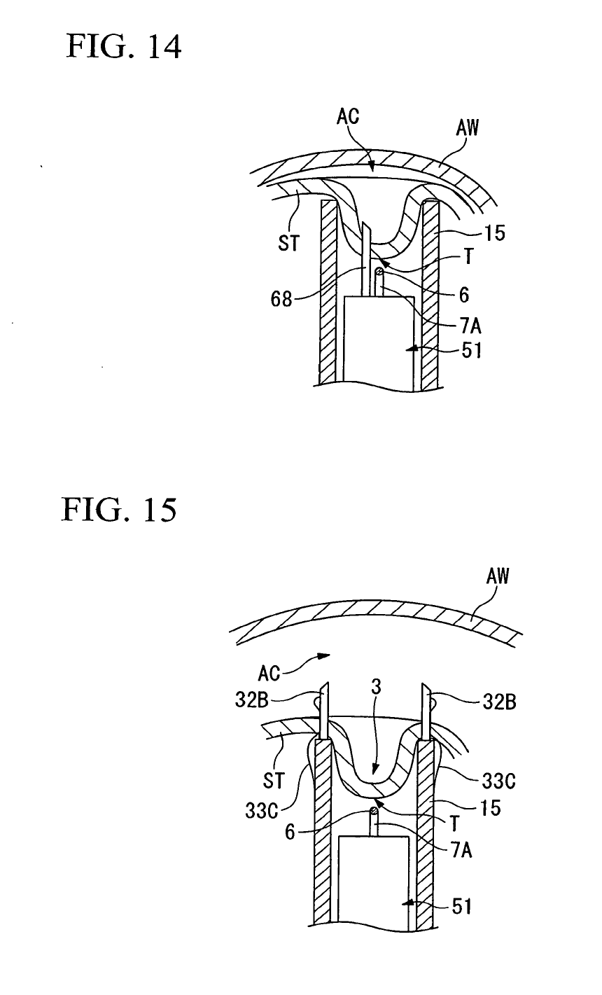

An abdominal cavity insufflating step (S42) is then performed. First, an injection needle 68 connected to the air/water feeding device 62 is inserted through the channel 60 of the endoscope 2. A distal end of the injection needle 68 is then protruded inside the distal end part 15, and as shown in FIG. 14, pierced through the suctioned stomach wall and inserted to the abdominal cavity AC. Because the injection needle 68 is pierced with the stomach wall being sucked in and a space being secured with respect to the abdominal cavity AC, just the stomach wall can be punctured reliably. Air is then fed into the abdominal cavity AC via the injection needle 68 to insufflate the abdominal cavity AC so that the stomach ST and the abdominal cavity AC separate.

The injection needle 68 preferably has a needle length of approximately 12 mm and more preferably has a bendable distal end to enable piercing of the center of the suctioned stomach wall. In this case, a bended injection needle has a bending tendency at a distal end and has a bending wire (not shown) that passes from the distal end toward a proximal side in an inward radial direction of the bending tendency. Here, since the channel 60 of the endoscope 2 is disposed at a position of six o'clock to eight o'clock of the endoscope inserting part 51, the incision site is approached from an upward angle in incising the anterior stomach wall of the stomach ST that is preferable as the incision site. Since the bending wire thus faces the center due to the bending tendency following the bended state of the insertion part 5 of the overtube 1, the center of the stomach wall can be punctured reliably by pulling the bending wire toward the proximal side. In the process of feeding air, the interior of the abdominal cavity AC may be maintained at an appropriate pressure by monitoring and automatic control of the feed air pressure.

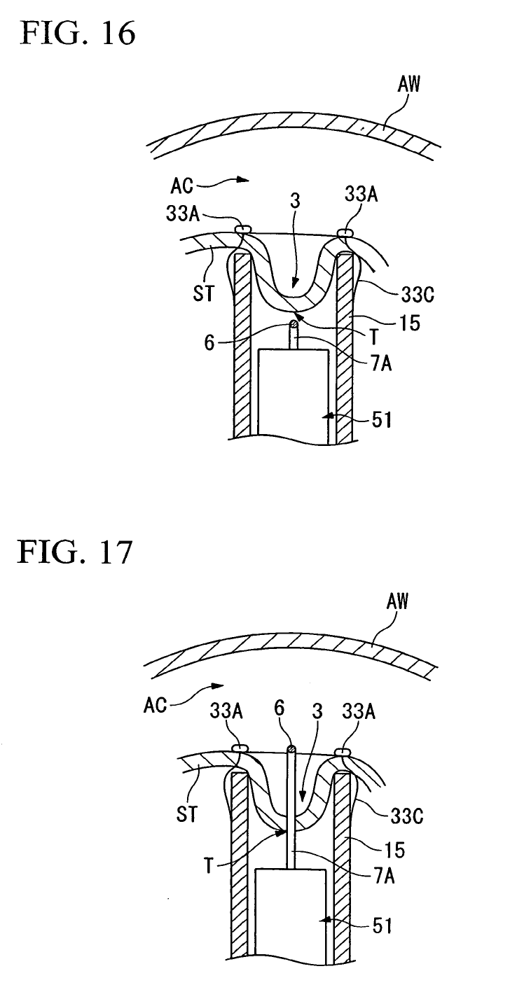

A placing step (S43) is then performed. First, the needle manipulating handle 41 is advanced in the direction of the sheath holding part 40 while holding the sheath holding part 40 to make the puncture needles 32A and 32B protrude from the first outer groove 30 and the second outer groove 31, respectively, of the distal end part 15 and pierce the stomach wall as shown in FIG. 15. From this state, the pusher connection part 43 is advanced with respect to the needle manipulating handle 41 to move the pushers 35 toward the distal ends of the puncture needles 32A and 32B. In this process, the anchors 33A of the double T-bars 33 are pressed by the pushers 35 and delivered out from inside the puncture needles 32A and 32B and into the interior of the abdominal cavity AC as shown in FIG. 16. Here, since the hole 37 is formed so that it is directed from the proximal end side toward the distal end side of the insertion part 5, unintended falling off of the stopper 33B of the double T-bars 33 is prevented. Here, since the abdominal cavity AC is insufflated to secure a space with respect to the stomach wall, just the stomach can be punctured.

After the anchors 33A of the double T-bars 33 have been released, the pusher connection part 43 is retracted with respect to the needle manipulating handle 41, and furthermore, the needle manipulating handle 41 is retracted with respect to the sheath holding part 40 to respectively accommodate the puncture needles 32A and 32B inside the first outer groove 30 and the second outer groove 31 again. In this process, the two anchors 33A of the double T-bars 33 are put in a T-like state by the bending tendencies of the sutures 33C. Thereafter, by holding and drawing the sheath holding part 40 towards the proximal side, the puncture needles 32A and 32B are removed from the distal end part 15 and by furthermore drawing the puncture needles out from the overtube 1, the bending property of the bending part 13 is secured.

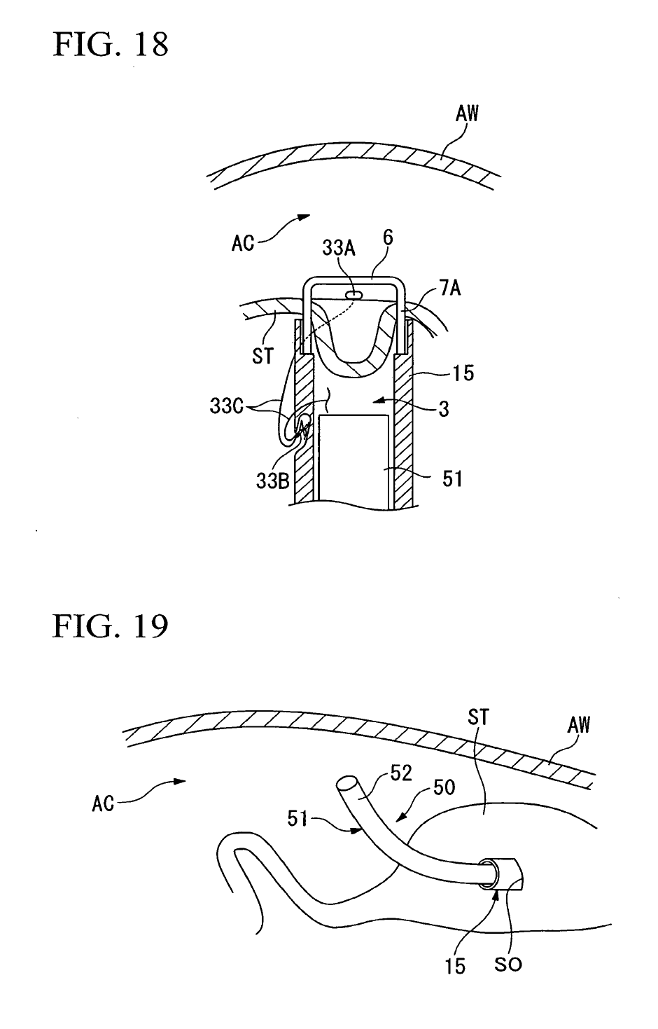

An incising step (S50) is then performed. First, it is checked whether the connection terminal 26A of the electrode manipulating part 8 is connected to the connection terminal 26B of the power supply cord 28. Then, while supplying the high-frequency power from high-frequency power supply 27, the manipulating handle 21 is advanced with respect to the manipulating body 20 to make the incising electrode 6 protrude from the distal end part 15 and contact the stomach wall. In this process, since the electricity is supplied to the incising electrode 6 via the electrode manipulating wires 7A and 7B, the stomach wall is incised by the incising electrode 6 and the opening SO is formed in the stomach wall as shown in FIGS. 17 and 18. By continuing the suctioning of the stomach wall in this step, the position of placement of the double T-bars 33 and the incision position are put in an optimal state.

A removing step (S60) is then performed. Here, in order to remove the incising electrode 6 from inside the insertion part 5, the fixing screw 24 of the manipulating body 20 of the electrode manipulating part 8 is loosened. In this process, the electrode manipulating wires 7A and 7B separate from the connection plate 23 and the electrode manipulating wires 7A and 7B become severed. Then, for example, an end part of the electrode manipulating wire 7A is held and drawn toward the proximal side to move the electrode manipulating wire 7A through the lumen 3 to the proximal end side and move the electrode manipulating wire 7B through the lumen 3 to the distal end side. Eventually, the electrode manipulating wire 7B also moves around the distal end opening of the lumen 3 and toward the proximal end side. The incising electrode 6 is thereby drawn out along with the electrode manipulating wires 7A and 7B.

An introducing step (S70) is then performed. That is, as shown in FIG. 19, the endoscope inserting part 51 of the endoscope 2, which is also an operative device, is introduced into the abdominal cavity AC through the opening SO. If, in this process, relative movement of the insertion part 5 and the endoscope inserting part 51 must be restricted, the endoscope lock button 47 is pressed and contacted against the endoscope inserting part 51 to fix the movement of the endoscope inserting part 51 by the frictional force. Since the endoscope lock button 47 is provided, the endoscope lock button 47 can be manipulated to restrain relative movement of the endoscope 2 with respect to the overtube 1, and the overtube 1 and the endoscope 2 can thus be inserted into the body simultaneously. Also, since the task of inserting the endoscope 2 can be performed while holding the proximal handle 44 of the overtube 1, an operation, in which the insertion part 5 of the overtube 1 is supported by one hand of the operator and the proximal handle 44 is held by the other hand, is enabled, and the operability is thus more improved.

When the overtube 1 is introduced into the abdominal cavity AC through the opening SO, the site of placement of the anchors 33A of the double T-bars is set at the proximal side of the position of the hole 37 formed in the insertion part 5. The stopper 33B, accommodated inside the hole 37, is thus pulled in the direction to become detached from the hole 37 in accordance with the orientation of the hole 37 and the stopper 33B falls out of the hole 37.

After positioning, a treating step (S80) of performing observation, incision, cell sampling, suturing, or any of other various treatments (medical procedures) is carried out. After performing the treatment, the overtube 1 and the endoscope 2 are removed from the opening SO of the stomach wall.

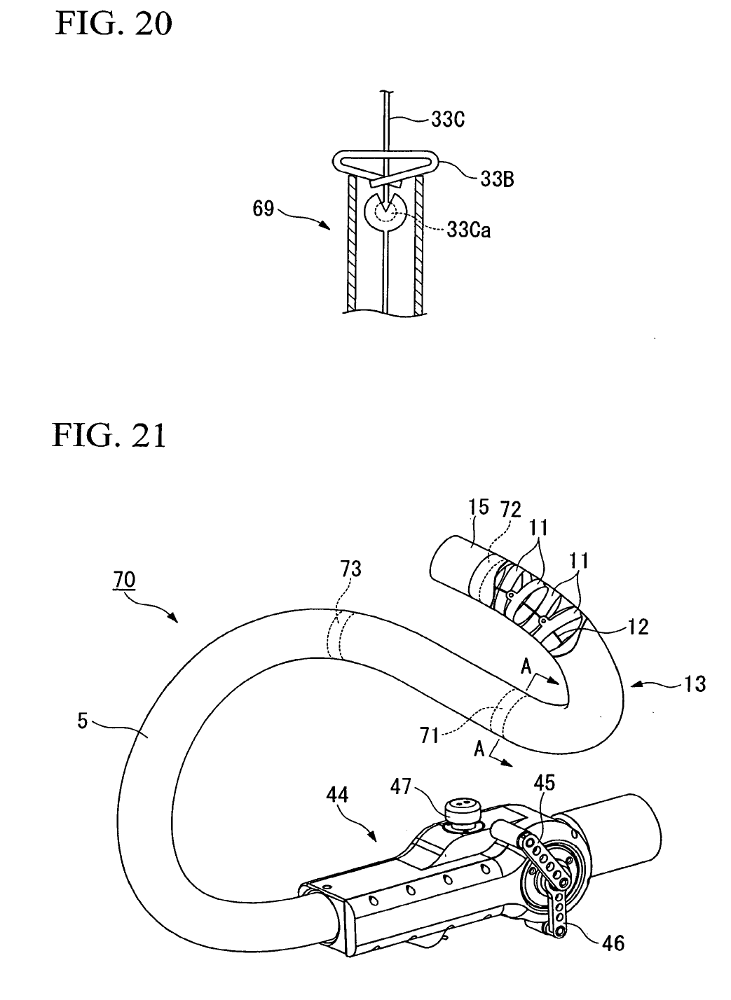

In a suturing step (S90), in removing the endoscope 2 from the opening SO, the large diameter part 33Ca of the sutures 33c is held and pulled with respect to the stopper 33B of the double T-bars 33, which had been placed in advance, by a ligating device 69, inserted through the channel 60 of the endoscope 2 as shown in FIG. 20. The opening SO is thereby sutured. Additional double T-bars 33, etc., are provided to perform further suturing if necessary. In this process, since the insufflation is performed in the process of placing the double T-bars 33 at the stomach wall, suturing by means of additional double T-bars 33 can be performed readily.

After suturing, the endoscope 2 is drawn out of the patient, the pressure applied to the abdominal cavity AC is released, and the surgical procedure is ended.

With the overtube 1, since the incising electrode 6 is disposed at the distal end side of the insertion part 5 and across the distal end side of the lumen 3, when the insertion part 5 is inserted into the stomach ST, the stomach wall can be incised without requiring a special treatment instrument for incision. Because, in this process, tissue is incised just by an amount corresponding to the length of the incising electrode 6 that crosses the lumen 3, the overtube 1 can be made to pass through with a light force and leakage at the outer periphery of the overtube 1 can be restrained preferably. Also, since the electrode manipulating wires 7A and 7B are removable with respect to the electrode manipulating part 8, the incising electrode 6 can be removed along with the electrode manipulating wires 7A and 7B from the insertion part 5. Thus, in making the endoscope 2 protrude out from the lumen 3, the incising electrode 6 will not be an obstruction, and upon inserting the endoscope 2 through the lumen 3, the endoscope 2 can be advanced into the abdominal cavity AC beyond the incised tissue. Furthermore, in making the device (endoscope 2 in the embodiment) that has been inserted through the lumen 3 protrude and advance from the distal end of the overtube 1 after incising tissue and forming the opening, the task of withdrawing the incising electrode 6 from the path of the device, the task of drawing out the overtube 1 once from within the body to remove the incising electrode 6, etc., can be omitted. Consequently, the surgical procedure time from the forming of the opening in the stomach wall to the introducing of the endoscope 2 into the abdominal cavity AC can be shortened.

Also, since the incising electrode 6 is connected to the electrode manipulating wires 7A and 7B, which can be manipulated to advance and retract with respect to the lumen 3, the incising electrode 6 can be advanced and retracted with respect to the lumen 3 without performing a manipulation of advancing and retracting the entirety of the insertion part 5. That is, by advancing and retracting of the electrode manipulating part 8, the incising electrode 6 can be advanced and retracted with respect to the stomach wall to perform incision. In this process, because the incision is performed by passing high-frequency electricity through the incising electrode 6, the incision can be performed more safely with a small force.

Also, before incising and forming an opening in a wall of a hollow organ (the stomach wall in the embodiment), the double T-bars 33 can be placed, and in inserting the puncture needles 32A and 32B through the stomach wall, puncture can be performed preferably without the stomach wall moving away. Furthermore, the double T-bars 33 can be placed before opening formation (before suturing) to set up a state in which the double T-bars 33 are just constricted in the suturing process, and in suturing the opening after ending the medical procedure inside the abdominal cavity AC, the suturing of the opening can be performed more readily without insufflating the interior of the stomach. The suturing task can thus be performed more readily.

Also, because the direction in which the incising electrode 6 crosses the lumen 3 is orthogonal to the direction of joining the puncture needles 32A and 32B, the positions of puncturing by the puncture needles 32A and 32B can be separated from the incision location, and the double T-bars 33 can be placed at a position separated from the incision location by a distance that is appropriate for binding.

### Second Embodiment

A second embodiment according to this invention shall now be described with reference to the drawings.

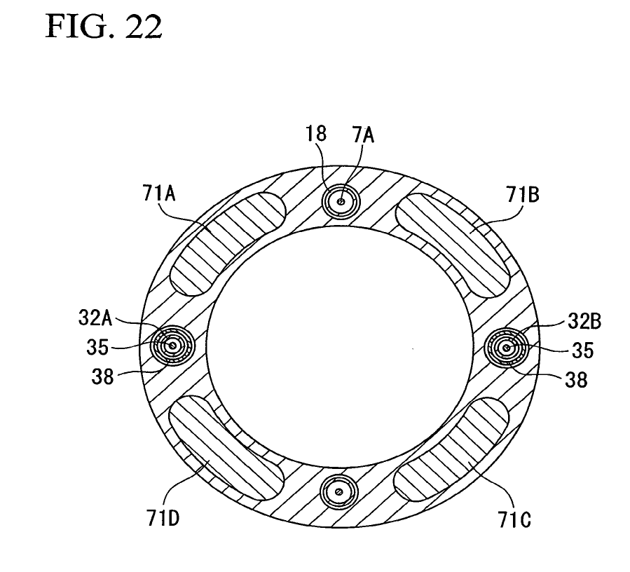

A point of difference of the second embodiment with respect to the first embodiment is that an overtube 70 according to this embodiment has a first magnet (magnetic body) 71, disposed on an outer peripheral surface of the insertion part 5 near the proximal end of the bending part 13, a second magnet (magnetic body) 72, disposed on an outer peripheral surface at the distal end of the bending part 13, and a third magnet (magnetic body) 73, disposed more toward the proximal end side (manipulating handle 21 side) of the insertion part 5 than the first magnet 71, as shown in FIG. 21. In order to restrain the insertion part 5 of the overtube 1 from becoming large in diameter and yet secure the inner diameter of the lumen 3, the first magnet 71 (and likewise, the second magnet 72 and the third magnet 73) is, for example, divided into and disposed as magnet pieces 71A, 71B, 71C, and 71D at portions besides portions at which the electrode tube 18 and the outer sheaths 38, inserted through the insertion part 5, are disposed as shown in FIG. 22.

Each of these magnets 71, 72, and 73 is formed so that all of the outer peripheral surface is of the same magnetic pole and these magnets are arranged so that the magnetic poles alternate along the insertion part 5, for example in a manner such that when the first magnet 71 is of the S pole, the second magnet 72 and the third magnet 73 are of the N pole.

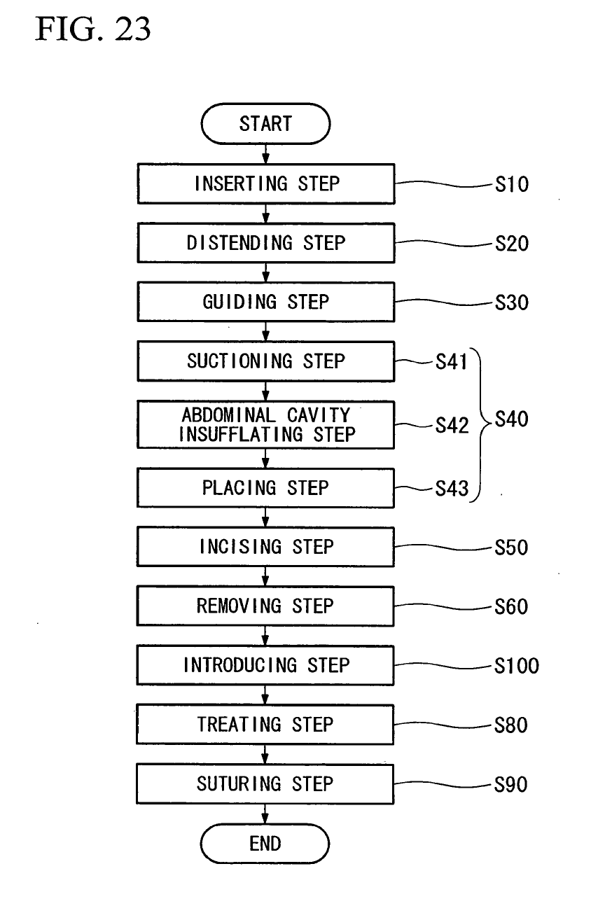

Actions of this embodiment shall now be described in line with a medical procedure performed via a natural orifice using the overtube 70 as shown by the flow chart of FIG. 23.

As in the first embodiment, the steps from the inserting step (S10) to the removing step (S60) are carried out in this embodiment as well.

An introducing step (S100) is then performed. That is, as shown in FIG. 19, the endoscope inserting part 51 of the endoscope 2 is introduced inside the abdominal cavity AC through the opening SO as shown in FIG. 19.

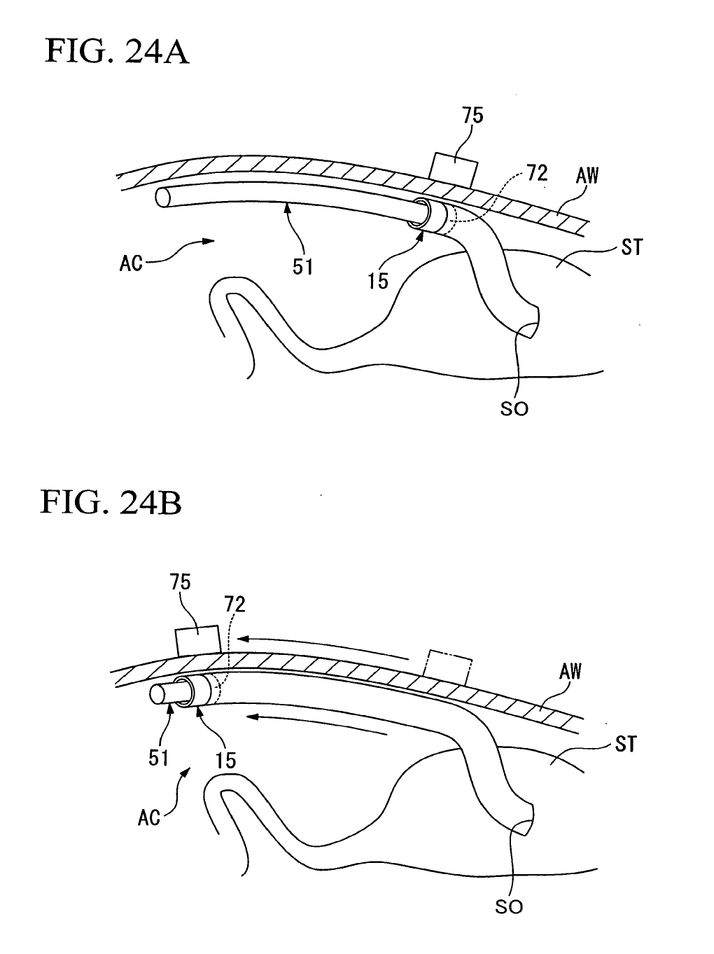

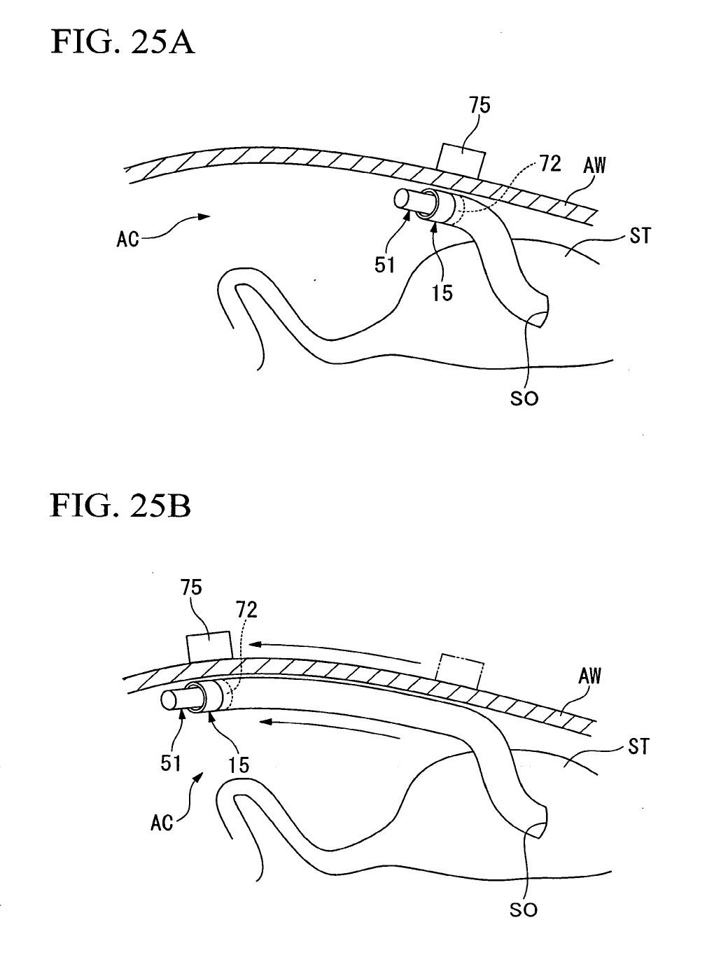

Then with the distal end part 15 of the overtube 70 being protruded from the opening SO of the stomach ST, a moving magnet 75 is placed on an abdominal wall AW near the opening SO with the magnetic pole that is attracted to the second magnet 72 of the overtube 70 being set at the inner side as shown in FIGS. 24A and 24B. In this process, the moving magnet 75 and the second magnet 72 are attracted to each other. The moving magnet 75 is then moved along the abdominal wall AW to a position at which a treatment site is located. In this process, the distal end 15 moves while being attracted to the moving magnet 75. The endoscope inserting part 51 may be advanced with respect to the insertion part of the overtube 70 in advance as shown in FIG. 24A, and then the moving magnet 75 may be used to advance the distal end part 15 of the overtube 70 along the endoscope inserting part 51 toward the distal end of the endoscope inserting part 51 as shown in FIG. 24B. Or, the moving magnet 75 may be moved with the endoscope inserting part 51 being accommodated inside the overtube 70 as shown in FIG. 25A, and then the endoscope inserting part 51 may be moved along with the overtube 70 as shown in FIG. 25B.

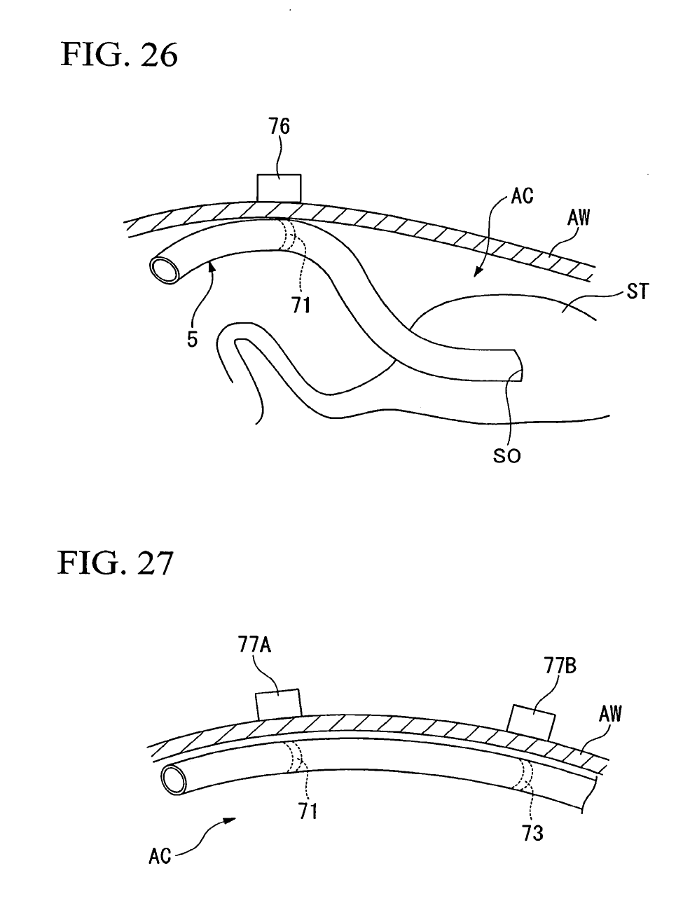

In order to secure a bended state of the bending part 13, a fixing magnet 76 is placed on the abdominal wall AW with the magnetic pole attracted to the first magnet 71 being set at the inner side as shown in FIG. 26. Since the fixing magnet 76 and the first magnet 71 are attracted to each other, a bending manipulation is performed by manipulating the bending lever 45 with the insertion part 5 being fixed to and supported on the abdominal wall AW. Here, in order to change the direction of the distal end part 15 with the bended state of the inserted part 5 being maintained, supporting magnets 77A and 77B are placed on the abdominal wall AW. That is, the supporting magnet 77A and the first magnet 71 are made to be attracted to each other, and the supporting magnet 77B and the third magnet 73 are made to be attracted to each other. When the mutual attraction state is realized, for example, the first magnet 71 side is rotatingly moved about the third magnet 73 side with the third magnet 73 side being fixed to change the direction of the distal end part 15 as shown in FIG. 27.

After then carrying out the treating step (S80), the endoscope 2 is returned into the stomach ST from the opening SO of the stomach wall and taken out from the mouth M of the patient PT, and then the suturing step (S90) is performed. The opening SO of the stomach wall is then sutured.

After suturing, the endoscope 2 is drawn out of the patient, the pressure applied to the abdominal cavity AC is released, and the surgical procedure is ended.

With the overtube 70, the same actions and effects as those of the first embodiment can be exhibited. In particular, since the first magnet 71, the second magnet 72, and the third magnet 73 are disposed at the outer portions of the insertion part 5, these can be attracted to the moving magnet 75, the fixing magnet 76, and the supporting magnets 77A and 77B to thereby support the insertion part 5 on the abdominal wall AW. The endoscope 2 that has been inserted into the overtube 70 can thus be restrained preferably from moving away during treatment. Also, by movement of the moving magnet 75, the distal end direction of the endoscope 2 that has been inserted into the overtube 70 can be moved readily and the direction of endoscope 2 can be controlled readily by the magnets. Also, movement, fixing, and supporting of the overtube 70 can be performed from outside the body by using the moving magnet 75, the fixing magnet 76, and the supporting magnets 77A and 77B to further facilitate orientation of the overtube 70.

The scope of the art of this invention is not restricted to the embodiments described above, and various changes can be added within a range that does not fall outside the spirit of this invention.

For example, though in each of the above-described embodiments, a flexible endoscope is used as the observation device, this invention is not restricted thereto and, for example, a so-called capsule endoscope may be placed inside the body, and while observing the interior of the body using the endoscope, an insertion part of a treatment device that does not have an observation device may be inserted through the overtube to perform the desired surgical procedure.

Though in the first embodiment, the incision electrode 6 is set to a length such that it is accommodated inside the lumen 3 in a bended state as shown in FIG. 4, this invention is not restricted thereto, and the incision electrode 6 may be set to a length such that it is accommodated inside the lumen 3 without being bended. The length of the incision electrode (length of the portion that crosses the lumen) may be set suitably according to the outer diameter of the overtube itself or according to the outer diameter of a device that is inserted through the lumen. This prevents the forming of an opening that is greater than necessary. Thus when a device is introduced into the abdominal cavity through an opening in a hollow organ that has been formed using the incision electrode, the gap formed between the device and the opening can be held at the minimum and sealing of the inner side and the abdominal cavity side of the hollow organ can be secured at a level by which the pressure applied to the abdominal cavity AC can be maintained.

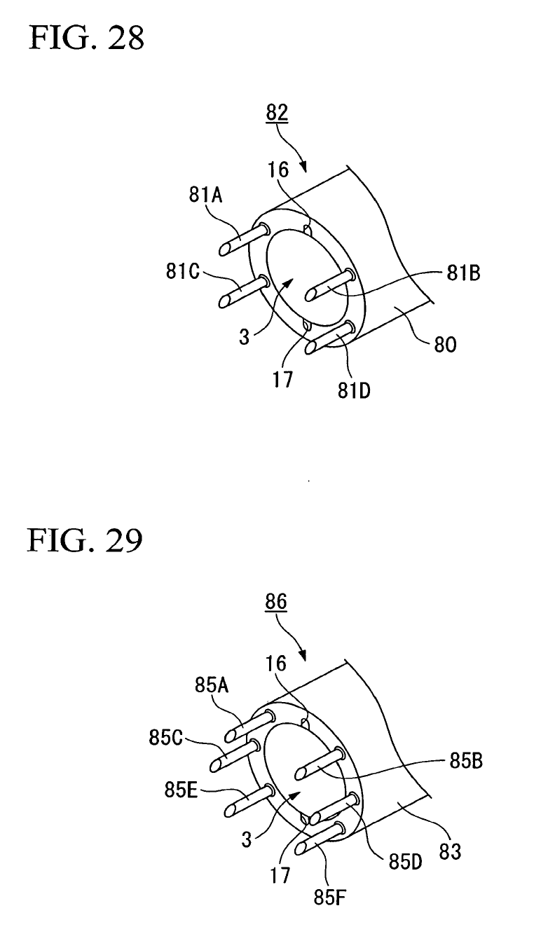

An overtube 82, having four puncture needles 81A, 81B, 81C, and 81D disposed at a distal end part 80, may be arranged as shown in FIG. 28. In this case, the puncture needles 81A and 81B and the puncture needles 81C and 81D are positioned at respectively symmetrical positions with respect to a line joining the first inner groove 16 and the second inner groove 17 of the distal end part 80. By housing the anchors 33A of the double T-bars 33 in the respective puncture needles, two suturing locations can be secured with respect to the incision direction.

Likewise, an overtube 86, having six puncture needles 85A, 85B, 85C, 85D, 85E, and 85F disposed at a distal end part 83, may be arranged as shown in FIG. 29. In this case, the puncture needles 85A and 85B, the puncture needles 85C and 85D, and the puncture needles 85E and 85F are positioned at respectively symmetrical positions with respect to a line joining the first inner groove 16 and the second inner groove 17 of the distal end part 83. By housing the anchors 33A of the double T-bars 33 in the respective puncture needles, three suturing locations can be secured with respect to the incision direction.

As shown in FIGS. 28 and 29, by providing four or six puncturing needles at the distal end part and thereby securing a plurality of suturing locations with respect to the incision location, a more reliable suturing can be carried out.

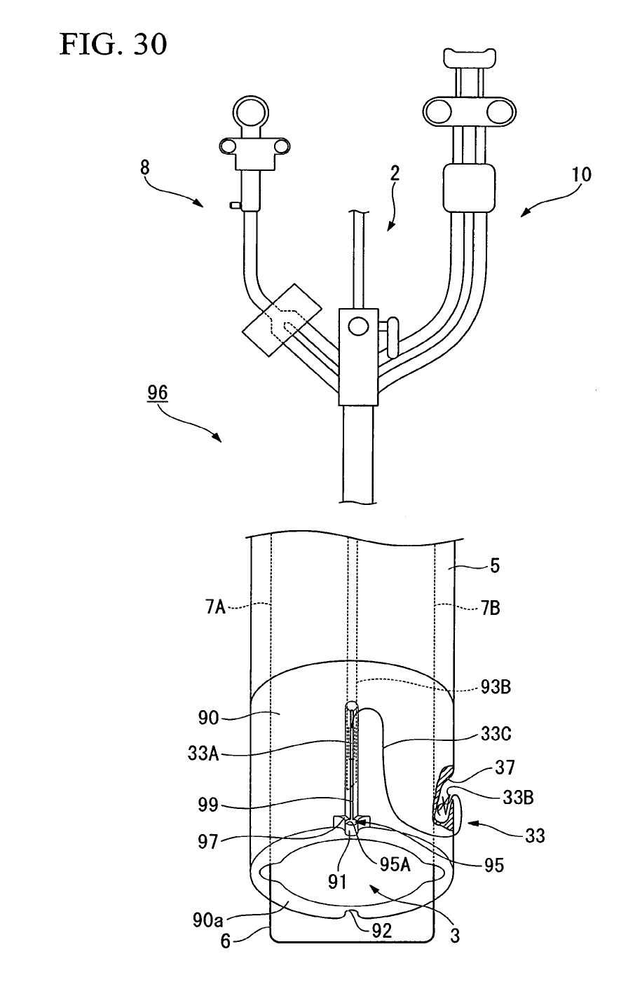

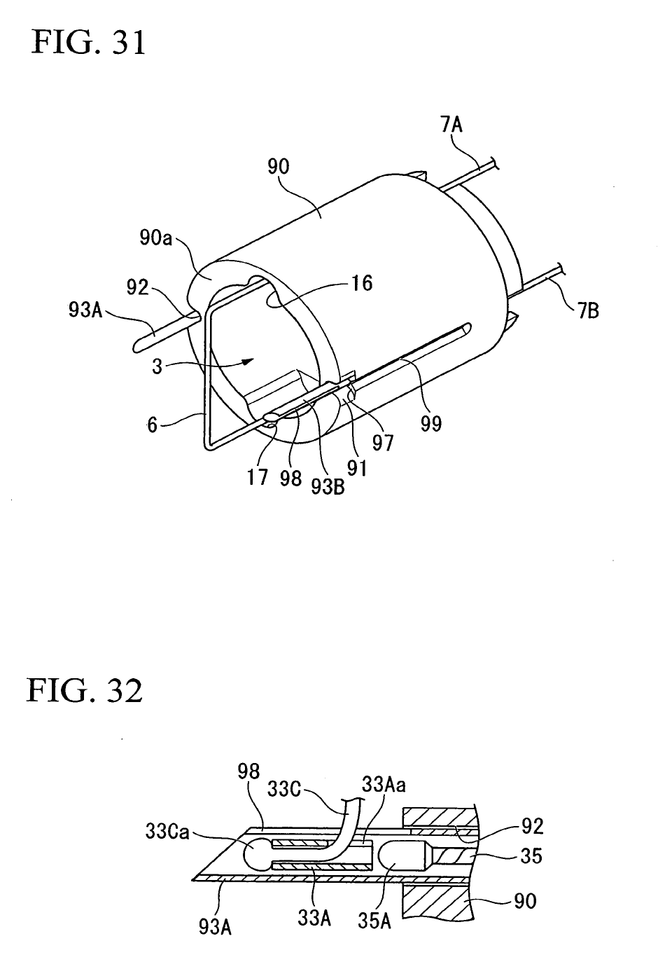

As shown in FIGS. 30 and 31, it is acceptable to provide an overtube 96 in which the opening 95A of a lumen for needle 95 is provided at the base end of a first outer groove 91 and a second outer groove 92 which are provided in the distal end part 90, the lumen for needle 95 extending from the base end side of the distal end part 90 and permitting retraction and projection of the puncture needles 93A, 93B. The inner diameter of the lumen for needle 95 is formed to be smaller than the width of the first outer groove 91 and the second outer groove 92, and to have a stepped portion 97. Furthermore, a first slit 98, into which the suture 33C of the double T-bar 33 can be inserted and passed through is provided extending in the longitudinal direction from the distal end of the puncture needles 93A and 93B, and a second slit 99, which is in communication with the outside surface of the distal end part 90 and into which the suture 33C can be inserted and passed through, is provided extending in the longitudinal direction from the opening 95A of the lumen for needle 95.

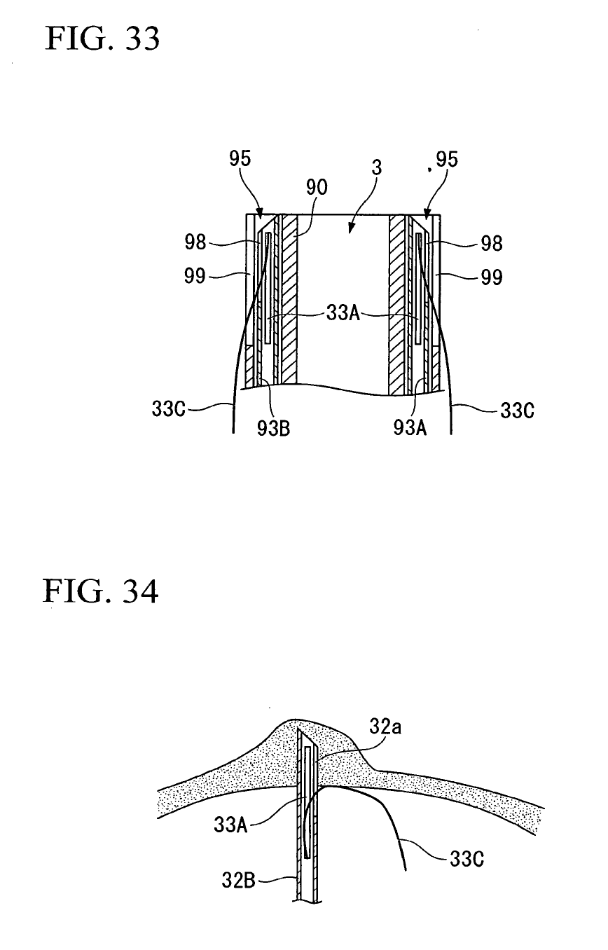

When housing the anchor 33A for the double T-bar 33 in the puncture needles 93A,93B, the anchor 33A is housed inside the puncture needles 93A, 93B, and stopper 33B is housed in the hole 37 by taking the suture 33C around the outer peripheral surface of the distal end part 90. Here, one end of the suture 33C is inserted into the anchor 33A, and is made to extend out so as to fold over from the slit 33Aa, with the stopper 33B being disposed at the other end. Since the suture 33C is formed, for example, of a resin such as nylon which is more highly elastic than thread or silk, the anchor 33A and the suture 33C are connected such that, in the natural state where there is no external force being applied, the suture 33C does not extend out in a perpendicular direction from the slit 33Aa of the anchor 33A, but rather forms an acute angle with respect to one end of the anchor 33A and forms an obtuse angle with respect to the other end of the anchor 33A. For this reason, when housing the anchor 33A in the puncture needles 93A, 93B, the direction of the anchor 33A is disposed so that the large diameter part 33Ca is directed toward the distal end of the puncture needles 93A, 93B, as shown in FIGS. 32 and 33. In this case, the suture 33C extends in the direction forming an acute angle with the base end side of the puncture needles 93A, 93B. Note that the anchor and the suture may be formed in a unitary manner of an identical material, such as nylon, for example. This suture 33C extends still further toward the outer surface of the distal end part 90 via the first slit 98 and the second slit 99. In this state, the puncture needles 93A,93B are retracted back toward the base end side of the overtube 96, with the distal ends of the puncture needles 93A, 93B housed so as to recede from the opening 95A of the lumen for needle 95.

Theoretically, in the case where there is no first slit 98, then suture 33C is exposed from the opening 95A of the lumen for needle 95, and extends out toward the outer surface of the distal end part 90, and the suture 33C is folded over inside the lumen for needle 95 at an acute angle. In this case, an undesirable bending tendency in the suture 33C may arise depending on the suture material used. When this bending tendency arises in the penetrating direction of the puncture needles 93A, 93B, then, as shown in FIG. 34, when the portion of the suture material in which bending has occurred comes into contact with the tissue, resistance is encountered. As a result, the puncturing operation does not go smoothly. Furthermore, theoretically, when the opening 95A of the lumen for needle 95 is on a side surface different from that of the hole 37, i.e., when the opening 95A is formed on the inside surface, then the suture 33C is disposed so as to transect the distal end surface 90a of the distal end part 90. Accordingly, when carrying out the placing step, the suture 33C becomes interposed between the distal end surface 90a and the tissue, and the suture 33C is pulled in the puncturing direction of the puncture needles 93A, 93B accompanying the action of puncturing the tissue with the puncture needles 93A, 93B. When the pulling force acts in a direction to move the tissue away from the distal end part 90, and when the pulling force is exceed a force that brings the tissue near and into contact with the distal end surface 90a, then the state in which the tissue is drawn to and held in contact with the distal end part 90 is released. In this case, it becomes difficult for the puncturing action to be carried out smoothly.

In contrast, by means of the present overtube 96, the opening 95A from which puncture needles 93A, 93B project out is formed to the same side as the outside of the distal end part 90. Thus, even if the tissue is drawn toward the distal end surface 90a, the suture 33C is not disposed between the distal end surface 90a and the tissue. Accordingly, the puncturing action can be carried out smoothly. Furthermore, since a first slit 98 is provided, it is possible to reduce the occurrence of undesirable bending in the suture 33C. Accordingly, the puncture operation can be carried out with greater certainty. In addition, by disposing the anchor 33A in puncture needles 93A, 93B as described above, the angle formed by the suture 33C and the tissue when puncturing the tissue is small, i.e., the angle formed between the direction of extension of the suture 33C and the lumen for needle 95 becomes closer to parallel. Accordingly, when puncturing, it is possible to limit the resistance of the suture 33C with respect to the tissue, and to carry out the puncturing operation with greater certainty.

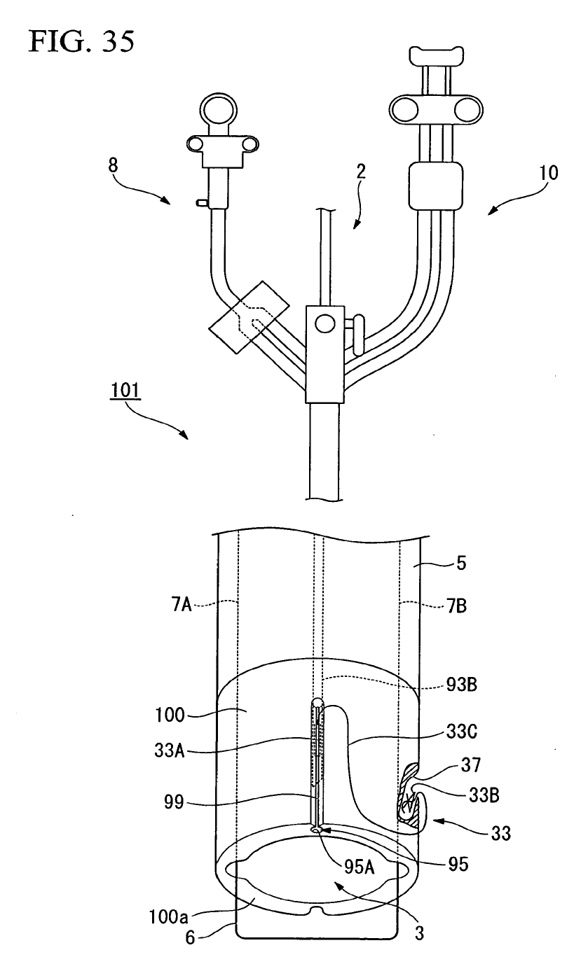

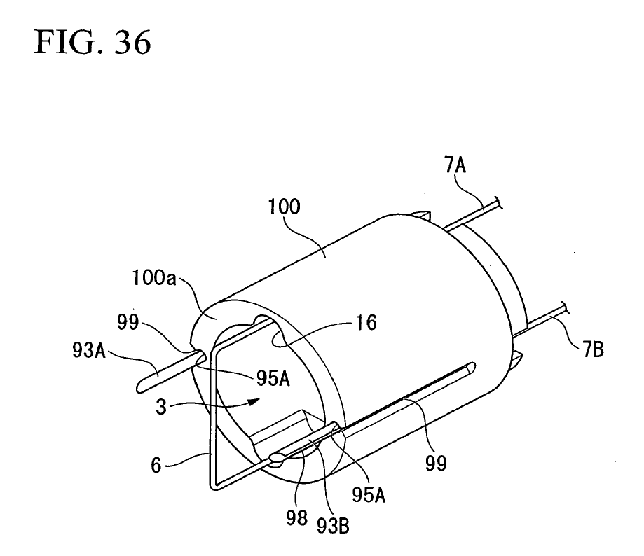

In addition, as shown in FIGS. 35 and 36, it is also acceptable to provide in place of a first outer groove 91 and a second outer groove 92, an overtube 101 in which a lumen for needle 95 is provided opening directly on the distal end surface 100a of the distal end part 100. This arrangement offers actions and effects equivalent to those of the overtube 101 described above.

What is claimed is:

1. A medical system comprising:
a flexible tube having a distal end portion and a proximal end portion and is provided with at least two magnetic bodies;
a medical device that is capable of sliding inside of the tube;
a moving magnet disposed outside of an abdominal cavity so as to be freely movable and capable of attracting the at least two magnetic bodies with a magnetic force;
wherein a positional relationship of the magnetic bodies inside of the body cavity is changed by changing the position of each of the magnetic bodies based on a moving distance of the moving magnet which is moved on the outside of the abdominal cavity so as to adjust a position or a direction of the flexible tube or the medical device in the body cavity.

2. The medical system according to the claim 1, wherein
the magnetic bodies are consisted of magnets, and the magnetic poles of the magnets are aligned on an outer periphery plane of the tube so as to allow the adjustment of the position or the direction of the flexible tube or the medical device in the body cavity even if a position of the tube is rotated.

3. The medical system according to the claim 1, wherein
the tube is provided with at least one bending portion which is capable of being bent inside the body cavity.

4. The medical system according to the claim 3, wherein
at least one lumen is disposed inside of an external wall of the tube; and the at least one lumen and the magnetic bodies are placed inside the external wall of the tube so as to be aligned on a substantially same circumference of the tube.

5. The medical system according to the claim 4 wherein:
at least two lumens are disposed inside the external wall of the tube, and the magnetic bodies are divided into at least two parts and disposed inside the external wall of the tube.

6. The medical system according to the claim 3, wherein
the at least two magnetic bodies are disposed on the proximal end side and the distal end side of the tube with respect to the bending portion of the tube.

7. The medical system according to the claim 3, wherein
the at least two magnetic bodies are disposed on the proximal end side of the tube with respect to the bending portion of the tube.
